(19) United States
(12) Patent Application Publication
(10) Pub. No.: US 2014/0271601 A1
(43) Pub. Date: Sep. 18, 2014

(54) CROSSLINKABLE TREHALOSE FOR THE COVALENT INCORPORATION IN HYDROGELS AND METHODS OF USE

(71) Applicants: Massachusetts Institute of Technology, Cambridge, MA (US); InVivo Therapeutics Corporation, Cambridge, MA (US)

(72) Inventors: Alex A. Aimetti, Waltham, MA (US); Timothy M. O'Shea, Cambridge, MA (US); Robert S. Langer, Newton, MA (US); Xueqing Zhang, Cambridge, MA (US)

(73) Assignee: InVivo Therapeutics Corporation, Cambridge, MA (US)

(21) Appl. No.: 14/209,697

(22) Filed: Mar. 13, 2014

Related U.S. Application Data
(60) Provisional application No. 61/779,506, filed on Mar. 13, 2013.

(51) Int. Cl. A61K 47/34; A61K 38/51

(52) U.S. Cl. CPC A61K 47/34; A61K 38/51
USPC 424/94.5; 514/772.3; 528/361; 527/314

(57) ABSTRACT

Methods of controlled delivery of bioactive therapeutics are provided. Compositions comprising therapeutic implants are provided.

### CROSS REFERENCE TO RELATED APPLICATION

This application claims the benefit of U.S. provisional patent application No. 61/779,506, which was filed Mar. 13, 2013 and is incorporated herein by reference as if fully set forth.

### FIELD

This disclosure relates to crosslinkable trehalose, covalent incorporation of trehalose in hydrogels, and methods of use thereof.

### BACKGROUND

Hydrogels represent a class of biomaterials that have received growing interest in the medical device industry, particularly in the fields of drug delivery and regenerative medicine. These materials are comprised of crosslinked polymer networks rendering the device insoluble. In addition, hydrogels are made up primarily of water which gives rise to desirable mechanical properties for use in vivo. Hydrogels have been used extensively as controlled drug delivery vehicles where a range of therapeutics (small molecules, growth factors, enzymes, antibodies, RNA, DNA, etc.) can be incorporated within the hydrogel via physical entrapment or chemical modification.

Synthetic polymers have been explored as desirable precursor molecules for use within hydrogels due to the high control of resulting chemical and mechanical material properties. However, there is interest in exploiting inherent advantages of natural products within hydrogel matrices. Modifying these molecules with specific functional groups allows for the facile incorporation within hydrogel materials as a means to engineer unique properties with the matrix.

### SUMMARY

In an aspect, the invention relates to a composition. The composition comprises a trehalose containing hydrogel; and a therapeutic.

In an aspect, the invention relates to a trehalose containing hydrogel. The trehalose containing hydrogel comprises a first polymer, and a second polymer. At least one of the first polymer or the second polymer includes trehalose covalently bound thereto.

In an aspect, the invention relates to a composition. The composition includes a first polymer component and a second polymer component. The first polymer component includes nucleophilic functional groups, a degree of functionality of greater than or equal to two, and is selected from the group consisting of a branched monomer, a multifunctional monomer, a branched polymer, and a multifunctional polymer. The second polymer component includes electrophilic functional groups, a degree of functionality of greater than or equal to two and selected from the group consisting of a branched monomer, a multifunctional monomer, a branched polymer, and a multifunctional polymer. At least one of the first polymer component or the second polymer component includes crosslinkable trehalose.

In an aspect, the invention relates to a composition. The composition includes a trehalose containing hydrogel. The hydrogel includes at least one of a natural or synthetic polymer.

In an aspect, the invention relates to a kit. The kit comprises a first vessel containing a first polymer component having nucleophilic functional groups, a degree of functionality of greater than or equal to two, and is selected from the group consisting of a branched monomer, a multifunctional monomer, a branched polymer, and a multifunctional polymer. The kit also comprises a second vessel containing a second polymer component having electrophilic functional groups, a degree of functionality of greater than or equal to two and selected from the group consisting of a branched monomer, a multifunctional monomer, a branched polymer, and a multifunctional polymer. At least one of the first polymer component or the second polymer component includes crosslinkable trehalose.

In an aspect, the invention relates to a composition. The composition comprises a sugar containing hydrogel; and a therapeutic.

In an aspect, the invention relates to a sugar containing hydrogel. The sugar containing hydrogel comprises a first polymer, and a second polymer. At least one of the first polymer or the second polymer includes sugar covalently bound thereto.

In an aspect, the invention relates to a composition. The composition includes a first polymer component and a second polymer component. The first polymer component includes nucleophilic functional groups, a degree of functionality of greater than or equal to two, and is selected from the group consisting of a branched monomer, a multifunctional monomer, a branched polymer, and a multifunctional polymer. The second polymer includes electrophilic functional groups, a degree of functionality of greater than or equal to two and selected from the group consisting of a branched monomer, a multifunctional monomer, a branched polymer, and a multifunctional polymer. At least one of the first polymer component or the second polymer component includes crosslinkable sugar.

In an aspect, the invention relates to a composition. The composition includes a sugar containing hydrogel. The hydrogel includes at least one of a natural or synthetic polymer.

In an aspect, the invention relates to a kit. The kit comprises a first vessel containing a first polymer component having nucleophilic functional groups, a degree of functionality of greater than or equal to two, and is selected from the group consisting of a branched monomer, a multifunctional monomer, a branched polymer, and a multifunctional polymer. The kit also comprises a second vessel containing a second polymer component having electrophilic functional groups, a degree of functionality of greater than or equal to two and selected from the group consisting of a branched monomer, a multifunctional monomer, a branched polymer, and a multifunctional polymer. At least one of the first polymer component or the second polymer component includes crosslinkable sugar.

In an aspect, the invention relates to a method of controlled therapeutic delivery. The method includes implanting the trehalose containing hydrogel or precursors of the trehalose containing hydrogel at a treatment site in a patient in need thereof.

In an aspect, the invention relates to a method of controlled therapeutic delivery. The method includes implanting the sugar containing hydrogel or precursors of the sugar containing hydrogel at a treatment site in a patient in need thereof.

In an aspect, the invention relates to a method of making a therapeutic implant comprising: inserting a crosslinkable trehalose containing polymer within a polymeric hydrogel to form a crosslinked hydrogel; and incorporating a therapeutic in the crosslinked hydrogel to form the therapeutic implant.

In an aspect, the invention relates to a method of making a therapeutic implant comprising: inserting a crosslinkable sugar containing polymer within a polymeric hydrogel to form a crosslinked hydrogel; and incorporating a therapeutic in the crosslinked hydrogel to form the therapeutic implant.

In an aspect, the invention relates to a method of formulating a trehalose containing hydrogel. The method includes combining the components of the trehalose containing hydrogel and allowing the components to polymerize.

In an aspect, the invention relates to a method of formulating a sugar containing hydrogel. The method includes combining the components of the sugar containing hydrogel and allowing the components to polymerize.

### BRIEF DESCRIPTION OF THE DRAWINGS

The following detailed description of the preferred embodiments of the present invention will be better understood when read in conjunction with the appended drawings. For the purpose of illustrating the invention, there are shown in the drawings embodiments that are presently preferred. It is understood, however, that the invention is not limited to the precise arrangements and instrumentalities shown. In the drawings:

FIG. 1 illustrates a trehalose containing hydrogel and a method of obtaining the same.

FIG. 2 illustrates LC-MS of trehalose diacrylate.

FIG. 3 illustrates ¹H-NMR of trehalose diacrylate.

FIG. 4 illustrates LC-MS of trehalose dimethacrylate.

FIG. 5 illustrates LC-MS of trehalose tetrathiol.

FIG. 6 illustrates ¹H-NMR of trehalose tetrathiol.

FIG. 7 illustrates examination of hydrogels by FTIR. Hydrogels (20 wt % where all acrylates came from trehalose diacrylate) were formulated into small hydrogel discs of 60 uL volume and placed in PBS to equilibrate for 48 hours with the incubation buffer replaced once at 24 hours. The sample was then placed under high vacuum drying for 72 hours before than being examined by FTIR using an alpha-FTIR with ZnSe ATR crystal module.

FIG. 8 illustrates rheology characterization of trehalose containing hydrogels. Open/closed circles: trehalose diacrylate+4-arm PEG-SH (5 kDa). Open/closed triangles: trehalose diacrylate (50% by acrylates)+PEGDA575 (50% by acrylates)+4-arm PEG-SH (5 kDa).

FIG. 9 illustrates rheology of 20 wt % hydrogel (all acrylates coming from trehalose diacrylate) in varying buffer pH.

FIG. 10 illustrates hydrogel swelling. Trehalose diacrylate and PEGDA 575 was used at various ratios to form the total amount of acrylate groups. 20 wt % hydrogels were formed and allowed to equilibrate under conditions similar to that described for FIG. 8. Mass as cured and mass observed at 48 equilibration was used to assess the swelling ratio.

FIG. 11 illustrates stabilization of NT-3 using trehalose diacrylate (TDA) or trehalose dimethacrylate (TDMA).

FIG. 12 illustrates activity of HRP (200 μg/ml) after 2 hours incubation at 70° C., within solutions or hydrogels containing varying amount of trehalose. For solution samples, non-modified trehalose dehydrate was added to be equivalent to the molar amounts of trehalose diacrylate in their corresponding hydrogels. Data shown as the average of n=2 with standard deviation.

### DETAILED DESCRIPTION OF EMBODIMENTS

Certain terminology is used in the following description for convenience only and is not limiting.

As used herein, “bioactive” or “active” are used interchangeably and refer to a therapeutic molecule that has therapeutic action to a specific target, substrate, or receptor.

As used herein, “biological therapeutic” refers to a molecule comprising monomeric units found in nature or derivatives thereof, the monomeric units may be, but are not limited to, amino acids, nucleotides or saccharides and any combination thereof, that are bioactive.

As used herein, “crosslinkable trehalose” refers to a trehalose disaccharide that is chemically modified with either nucleophilic functional groups or electrophilic functional groups. The nucleophilic functional groups may be thiols. The electrophilic functional groups may be acrylates.

As used herein, a “trehalose containing hydrogel” is a hydrogel including trehalose crosslinked within the hydrogel or trehalose covalently bound to a polymer crosslinked in the hydrogel. A trehalose containing hydrogel may also be referred to herein as a crosslinked hydrogel.

As used herein, “a sugar containing hydrogel” is a hydrogel including sugars crosslinked within the hydrogel or sugars covalently bound to a synthetic or natural polymer crosslinked in the hydrogel. Sugars may include monosaccharides, disaccharides, oligosaccharides, or polysaccharides. Non-limiting examples of sugars that may be a sugar in a sugar containing hydrogel are sucrose, glucose, mannose, galactose, fructose, trehalose, or oligosaccharides containing a mixture of the said sugars. Non-limiting examples of polysaccharides include chitosan, dextran, alginate, hyaluronic acid, cellulose (non-modified or modified), amylose, chondroitin sulfate, and heparin sulfate.

As used herein, “site-specific modification” means that a chemical modification occurs in a designated position within a molecule.

As used herein “multifunctional” means that a molecule is modified with n reactive functional groups where n>2.

As used herein, “degree of functionality” describes the number of desired reactive functional groups within a molecule.

As used herein, “activated alkene” refers to a carbon-carbon double bond which is located near, or attached to, an electronegative substituent that causes the carbon-carbon double bond to become electron deficient and capable of acting as an acceptor in the Michael-addition reaction.

The words “a” and “one,” as used in the claims and in the corresponding portions of the specification, are defined as including one or more of the referenced item unless specifically stated otherwise. This terminology includes the words above specifically mentioned, derivatives thereof, and words of similar import. The phrase “at least one” followed by a list of two or more items, such as “A, B, or C,” means any individual one of A, B or C as well as any combination thereof.

In an embodiment, a sugar containing hydrogel is provided. Embodiments also include methods of use and methods of formation of a sugar containing hydrogel. Sugar containing hydrogels are exemplified herein by trehalose containing hydrogels, methods of use thereof, and methods formation thereof. Based on the description of trehalose containing hydrogels, methods of use, and methods of formation, the other sugar containing hydrogels and methods are described. The skilled artisan would recognize that the other sugar would be in place of the trehalose. Non-limiting examples of sugars that could be in the place of trehalose in the description below are sucrose, glucose, mannose, galactose, fructose, or oligosaccharides containing a mixture of the said sugars. Non-limiting examples of polysaccharides include chitosan, dextran, alginate, hyaluronic acid, cellulose (non-modified or modified), amylose, chondroitin sulfate, and heparin sulfate. A sugar containing hydrogel may include more than one type of sugar.

In an embodiment, a trehalose containing hydrogel is provided. The trehalose containing hydrogel may further comprise one or more type of biological therapeutic. A relatively large globular shape of a biological therapeutic may aid in incorporating the biological therapeutic in the trehalose containing hydrogel. Examples of biological therapeutics include, but are not limited to, proteins, antibodies, peptides, enzymes, RNA, DNA, vaccines, and viruses. The biological therapeutic may be physically entrapped within the crosslinked polymeric network of the trehalose containing hydrogel. The trehalose containing hydrogel may hinder, or control the rate of diffusion of the biological therapeutic agent outward from the hydrogel. For this reason, the hydrogel may be referred to as a depot. However, preventing thermal, hydrolytic, or proteolytic damage to maintain bioactivity and stability of a biological therapeutic for a prolonged time period remains a significant challenge. Trehalose is a naturally occurring disaccharide that has been shown to enhance and/or maintain the activity of biological therapeutics when added as an excipient in a formulation. See Stanwick et al., 2012 and Lee et al., 2010, cited below, which are incorporated herein by reference as if fully set forth. The exact mechanism for protein stabilization is unknown. However it is hypothesized to be one of the following: trehalose-protein interactions due to hydrogen bonding; sequestering of water molecules near the protein surface acting as a protectant; or trehalose-mediated entrapment of protein molecules. Embodiments herein include a novel use of a trehalose containing hydrogel, that may be formed from crosslinkable trehalose as a means to recapitulate the bioactivity stabilization effect in a 3-dimensional biomaterial environment for prolonged, controlled drug delivery applications. An embodiment includes a composition comprising the trehalose containing hydrogel. An embodiment includes a composition comprising natural or synthetic polymeric hydrogel, where the natural polymeric hydrogel may include as a polymer in the hydrogel peptides, monosaccharides, disaccharides, oligosaccharides, polysaccharides, or oligonucleotides; and the synthetic polymer hydrogel may include as a polymer in the hydrogel ethylene oxide, glycolide, lactide, or caprolactone repeat units. An embodiment includes a method of making a trehalose containing hydrogel.

An embodiment includes a method of controlled, active therapeutic delivery. The method may include inserting crosslinkable trehalose within a synthetic polymeric hydrogel to form a crosslinked hydrogel, and incorporating a therapeutic in the crosslinked hydrogel to form a therapeutic implant. The method may also include implanting the therapeutic implant. The therapeutic may be a biological therapeutic. Implanting may include physically placing the therapeutic implant to a treatment site in a patient. Physically placing may include surgically placing. Implanting may include administering hydrogel precursors, including crosslinkable trehalose, and the therapeutic to a treatment site in a patient. Administering precursors may include injecting the precursors at the treatment site. The treatment site may be, but is not limited to, a site of injury, or a site of disease. The treatment site may be the central nervous system, including the spinal cord and/or brain and its associated structures. The treatment site may be the peripheral nervous system, including a site within and on the spine, or the eye. The treatment site may be at a site of surgical intervention.

A method may include implanting a trehalose containing hydrogel. The trehalose containing hydrogel may include a therapeutic, which may be a biological therapeutic, and thus be a therapeutic implant. The method may include adding a therapeutic to the trehalose containing hydrogel. Implanting may include placing a formed trehalose containing hydrogel or therapeutic implant to a treatment site in a patient. Implanting may include administering hydrogel precursors, including crosslinkable trehalose, to a treatment site in a patient. Administering precursors may include injecting the precursors at the treatment site. The mixture of hydrogel precursors may include at least one therapeutic, which may be a biological therapeutic. The treatment site may be, but is not limited to, a site of injury, or a site of disease. The treatment site may be the central nervous system, including the spinal cord and/or brain and its associated structures. The treatment site may be the peripheral nervous system, including a site within and on the spine, or the eye. The treatment site may be at a site of surgical intervention.

As used herein, a patient is an animal. The animal may be a mammal. The mammal may be a human being. The patient may be in need of treatment. The patient may be suffering from spinal cord injury, tumor resection, intervertebral disc disease, lumbar disc disease, peripheral nerve injury, ocular indications, stroke, traumatic brain injury, aneurisms, or spinal fusion, and may be in need of treatment thereof. The patient may be in need of prophylaxis from injury or disease, and the therapeutic implant or trehalose containing hydrogel may achieve the prophylaxis. The prophylaxis may be by way of vaccine components as the biological therapeutic.

Crosslinkable Trehalose

Trehalose contains multiple hydroxyls for potential chemical modification. To site-specifically modify the primary hydroxyls in the 6 and 6′ positions, an enzyme catalytic approach may be taken. This reaction scheme can be utilized to achieve multifunctional trehalose. To modify trehalose with electrophilic groups, the esterification reaction, it is reacted with vinyl ester functionalized C═CO(C═O)R1 molecules in the presence of C. antarctica lipase B where R1 can be an acrylate, methacrylate, ethacrylate, acrylamide, maleimide, or vinyl sulfone. R1 may be an acrylate. See Reaction Scheme 1, below. In an embodiment, C. antarctica lipase B is attached to macroporous acrylic resin, and reactants are contacted with the resin. This one step reaction may result in a difunctionalized trehalose molecule.

[chemical structure]

[chemical structure]

In an embodiment of the general reaction shown in Reaction Scheme 1, the reaction would yield trehalose diacrylate. Alternative trehalose chemical modification can be afforded by first reacting the sugar with a stoichiometric excess of a double vinyl ester which on purification yields trehalose with a vinyl ester functionality at 6 and 6′ positions of the sugar which are available for subsequent reaction with other primary hydroxyl-containing molecules (Reaction Scheme 2). Double vinyl esters that may be used as R2. R2 may be (CH₂)₄ to give divinyl adipate or (CH₂)₈ to give divinyl sebacate. R2 may be (CH₂)ₙ where n=1-20, (CH₂CH₂O)ₙ, or may contain glycolide, lactide, or caprolactone repeat units, or peptides. To modify trehalose with nucleophilic groups, a second esterification reaction may be performed between trehalose divinyl ester and a primary hydroxyl-containing molecule also comprising a stronger nucleophile, R3. R3 may be a thiol. R3 may comprise a multifunctional nucleophile. R3 may comprise (SH)ₘ(CH₂)ₙ where m=1-10 and n=1-20. R3 may comprise a dithiol. Examples of primary hydroxyl containing thiol molecules are 2,3-dimercapto-1-propanol, 2-mercaptoethanol, and 1-thioglycerol. The reaction with trehalose divinyl ester and 2,3-dimercapto-1-propanol could yield a multifunctional (tetra functional in this embodiment) trehalose with thiol moieties.

Trehalose Containing Hydrogel

In an embodiment, a method includes mixing hydrogel precursors to form a crosslinked hydrogel. The precursors may be a first polymer component and a second polymer component. The precursors may be a first polymer component, a second polymer component and a third polymer component. The first polymer component may different than the second polymer component. The third polymer component may be different than the first polymer component or the second polymer component. A trehalose containing hydrogel herein may be formed by this method. As used herein, “polymer components” refer to the molecular precursors that will react to form a hydrogel. The polymer components may be crosslinkable trehalose, a crosslinkable sugar, a natural or synthetic polymer, a branched monomer, a multifunctional monomer, a branched polymer, or a multifunctional polymer.

The first polymer component may have a degree of functionality greater than or equal to two and be selected from the group consisting of a branched monomer, a multifunctional monomer, a branched polymer and a multifunctional polymer. The first polymer component may include nucleophilic functional groups. The first polymer component may be crosslinkable trehalose. The first polymer component may be thiol-modified trehalose. The first polymer component may be multi-arm poly(ethylene glycol) functionalized with terminal thiol groups. The first component may be ethoxylated-trimethylolpropan tri(3-mercaptopropionate) (ETTMP).

The second polymer component may have a degree of functionality greater than or equal to two and be selected from the group consisting of a branched monomer, a multifunctional monomer, a branched polymer and a multifunctional polymer. The second polymer component may include electrophilic functional groups. The second polymer component may be crosslinkable trehalose. The second polymer component may include electrophilic functional groups. The second polymer component may be a methacrylate functionalized trehalose, an ethacrylate functionalized trehalose, a maleimide functionalized trehalose, a vinyl sulfone functionalized trehalose or an acrylate functionalized trehalose. The second polymer component may also be a dimer, trimer or any other number of repeat trehalose units with terminal functionality of the polymer being either methacrylate, ethacrylate, maleimide, vinyl sulfone or acrylate. The second polymer component may be trehalose diacrylate. The second polymer component may be poly(ethylene glycol) diacrylate.

A third polymer component may be added to vary the amount of trehalose in the trehalose containing hydrogel. The third polymer component may have a degree of functionality greater than or equal to one and be selected from the group consisting of a branched monomer, a multifunctional monomer, a branched polymer and a multifunctional polymer. The third polymer component may contain electrophilic or nucleophilic functional groups. The third polymer component may contain ethylene oxide or ethylene glycol repeat units and either nucleophilic or electrophilic functional groups. The third polymer component may be poly(ethylene glycol diacrylate).

Branched monomers, multifunctional monomers, branched polymers, and multifunctional polymers may contain ethylene glycol, ethylene oxide, caprolactone, lactide, glycolide, hydroxyethyl methacrylate, acrylamide, or trehalose units.

In an embodiment, the first polymer component is ethoxylated-trimethylolpropan tri(3-mercaptopropionate), the second polymer component is trehalose diacrylate and the third component is PEG diacrylate.

In an embodiment, the first polymer component is ethoxylated-trimethylolpropan tri(3-mercaptopropionate), the second component is trehalose diacrylate, and the third component is 8-arm PEG vinyl sulfone (Mₙ˜10 kDa).

Mixing may include adding the first polymer component to the second component in stoichiometric equivalencies relative to functional groups. Mixing may include adding the first polymer component to the second polymer component and the third polymer component in stoichiometric equivalencies relative to functional groups. For example, the nucleophilic functional groups of the first polymer component may be equal to the electrophilic functional groups in the second polymer component. Alternatively, all polymer components are added together where the concentration of nucleophilic functional groups are in stoichiometric equivalence with electrophilic functional groups.

Mixing may include adding the first polymer component to the second polymer component in a buffering medium. Mixing may include adding the first polymer component to the second component and the third polymer component in a buffering medium. The buffering medium may have a pH of greater than seven. The buffering medium may have a pH of 7 to 9.5.

Mixing may include adding trehalose diacrylate and PEG-diacrylate to ethoxylated-trimethylolpropan tri(3-mercaptopropionate) in stoichiometric equivalence relative to acrylate and thiol concentrations.

The trehalose-containing hydrogel can be designed to cure in situ when delivered (or implanted) to a treatment site (e.g., a tissue). In situ curing describes the transition from a solution to a crosslinked material. Biological therapeutics may be incorporated within the prepolymer solution (mixture of precursors) and become physically entrapped upon crosslinking of the polymer components. Further, the trehalose containing hydrogel can be prefabricated with encapsulated biological therapeutics and subsequently dehydrated. This crosslinked gel may have utility to increase shelf-life and storage of sensitive biological therapeutics such as vaccines. This crosslinked gel can be delivered as a pre-formed implant or the biological therapeutic can be retrieved in vitro upon equilibration in an aqueous environment and subsequently delivered through injection. Methods of using the crosslinked hydrogel envision implanting these utilities. For example, a method may include equilibrating a pre-formed implant in an aqueous environment.

Hydro gels are crosslinked polymer networks formed by a step-growth or chain-growth polymerization mechanism. In an embodiment, trehalose diacrylate can form hydrogels alone, or in the presence of other co-polymers, by the radical polymerization of acrylate groups. Radical initiators (thermal, redox, or photo) can be used to initiate the polymerization reaction. Further, radical polymerizations of components with multifunctional reactive groups result in a hydrogel with a kinetic chain in the structure. If a multifunctional acrylate is polymerized via a radical-initiation mechanism to form a crosslinked gel, the resulting material will contain hydrophobic polyacrylate kinetic chains. Step-growth reactions proceed with at least an A-A monomer or polymer and a B-B monomer or polymer to form a (A-AB-B)n polymer. In an embodiment, the trehalose containing hydrogel or therapeutic implant is a poly(ethylene glycol) (PEG)-based hydrogels formed via the base-catalyzed Michael-type reaction between a sulfhydryl and an activated alkene, which may be an acrylate. PEG-diacrylate (Mₙ˜ranging from 500 to 1,000 g mol⁻¹) and ethoxylated-trimethylolpropan tri(3-mercaptopropionate) (ETTMP) (Mₙ˜1300 g mol⁻¹) are combined together in stoichiometric equivalence relative to thiol (nucleophile) and acrylate (electrophile) concentrations to initiate gelation.

A utility of crosslinkable trehalose as a component to include within a crosslinked hydrogel with entrapped biological therapeutics to enhance or prolong the activity is demonstrated herein. Soluble, native trehalose if included in the prepolymer solution would elute out of the hydrogel in a short time scale (minutes-hours) due to the high-degree of water solubility and small structure size (hydrodynamic radius) of the sugar. By modifying the sugar to allow for it to be covalently crosslinked within the matrix provides a mechanism whereby the molecule is inhibited from diffusing away and provides its protective effects for over a longer time-course than if it was free to elute from the hydrogel.

In an embodiment, trehalose diacrylate and PEG-diacrylate contribute the acrylate functionality to the hydrogel reaction while the ETTMP contributes the sulfhydryl functionality to the mixture. The concentration of acrylate may equal the concentration of sulfhydryl. One can tailor the ratio of trehalose diacrylate to PEG-diacrylate to achieve various gel properties with desirable protein protective effects. For example, FIGS. 8-10 demonstrate tailorable control of mechanical properties, gelation kinetics, and hydrogel swelling.

The crosslinked hydrogel may have a polymer weight percent between and including 5 to 40 percent. The crosslinked hydrogel polymer weight percent may be any value between 5 and 40 percent. The crosslinked hydrogel weight percent may have a value in a range between and including any two integer percents from 5 to 40. As used herein, “hydrogel weight percent” is the (mass of covalently conjugated or crosslinked components in a crosslinked hydrogel)/(mass of covalently conjugated or crosslinked components plus the mass of water in the hydrogel).

The therapeutic, which may also be referred to as a therapeutic agent, may be a small molecule, a growth factor, a protein, a peptide, an enzyme, an antibody, RNA, DNA, vaccine, virus. The therapeutic may be at least one therapeutic selected from the group consisting of chondroitinase ABC (chABC, 1-50 units/mL), arylsulfatase B (ARSB, 1-50 units/mL), neurotrophin-3 (NT-3, 1-1000 μg/mL), insulin, human growth hormone (HGH, 1-1000 μg/mL), bone morphogenic protein-2 (BMP-2, 1-1000 μg/mL) or related family of BMP's, nerve growth factor (NGF, 1-1000 μg/mL), brain-derived neurotrophic factor (BDNF), glial-cell-line derived neurotrophic factor (GDNF, 1-1000 μg/mL), hepatocyte growth factor (HFG, 1-1000 μg/mL), exozyme C3 transferase (Cethrin, 1-1000 μg/mL) and its derivatives, basic fibroblast growth factor (bFGF, 1-1000 μg/mL), acid fibroblast growth factor (aFGF, 1-1000 μg/mL), transforming growth factor b1 (TGF-β1, 1-1000 μg/mL), epidermal growth factor (EGF, 1-1000 μg/mL), platelet-derived growth factor (PDGF, 1-1000 μg/mL), insulin-like growth factor 1 (IGF-1, 1-1000 μg/mL), vascular endothelial growth factor (VEGF, 1-1000 μg/mL), leukemia inhibitory factor (LIF, 1-1000 μg/mL), and anti-Nogo antibody (1-100 mg/mL), myelin associated glycoprotein (MAG) antibody (1-100 mg/mL), oligodendrocyte myelin glycoprotein (OMgp) antibody (1-100 mg/mL), ephrin B3 antibody (1-100 mg/mL), semaphorins 4a/4d/6a antibody (1-100 mg/mL), netrin 1 antibody (1-100 mg/mL), repulsive guidance molecule A (RGMa) antibody (1-100 mg/mL), and erythropoietin (25,000-2,500,000 IU/mL). The methods and compositions herein may be applied with the biological therapeutics listed above or others for a variety of medical interventions. For a controlled protein delivery treatment regime, efficacy of the therapeutic may depend upon whether it remains active.

### EMBODIMENTS

The following list includes particular embodiments of the present invention. But the list is not limiting and does not exclude alternate embodiments, as would be appreciated by one of ordinary skill in the art.

1. A composition comprising:

a trehalose containing hydrogel; and

a therapeutic.

2. The composition of embodiment 1, wherein the trehalose containing hydrogel includes a natural or synthetic polymer.

3. The composition of any one or more of embodiments 1-2, wherein the trehalose containing hydrogel includes the product of step growth or chain growth polymerization between ethoxylated-trimethylolpropan tri(3-mercaptopropionate) and trehalose diacrylate.

4. The composition of any one or more of the preceding embodiments, wherein the trehalose containing hydrogel includes the product of step growth or chain growth polymerization between ethoxylated-trimethylolpropan tri(3-mercaptopropionate), trehalose diacrylate, and PEG diacrylate.

5. The composition of any one or more of the preceding embodiments, wherein the trehalose containing hydrogel includes the product of step growth or chain growth polymerization between ethoxylated-trimethylolpropan tri(3-mercaptopropionate), trehalose diacrylate, and an 8-arm PEG vinyl sulfone.

6. The composition any one or more of the preceding embodiments, wherein the trehalose is in the form of a trehalose repeat unit with n repeating units of trehalose and n is greater than or equal to two.

7. The composition of any one or more of the preceding embodiments, wherein the therapeutic is a biological therapeutic.

8. The composition of embodiment 7, wherein the biological therapeutic is selected from the group consisting of a growth factor, a protein, a peptide, an enzyme, an antibody, an RNA, a DNA, a vaccine, and a virus.

9. The composition of any one or more of embodiments 7-8, wherein the biological therapeutic is selected from the group consisting of chondroitinase ABC (chABC), arylsulfatase B (ARSB), neurotrophin-3 (NT-3), insulin, human growth hormone (HGH), bone morphogenic protein-2 (BMP-2) or related family of BMP's, nerve growth factor (NGF), brain-derived neurotrophic factor (BDNF), glial-cell-line derived neurotrophic factor (GDNF), hepatocyte growth factor (HFG), exozyme C3 transferase (Cethrin) and its derivatives, basic fibroblast growth factor (bFGF), acid fibroblast growth factor (aFGF), transforming growth factor b1 (TGF-β1), epidermal growth factor (EGF), platelet-derived growth factor (PDGF), insulin-like growth factor 1 (IGF-1), vascular endothelial growth factor (VEGF), leukemia inhibitory factor (LIF), and anti-Nogo antibody, myelin associated glycoprotein (MAG) antibody, oligodendrocyte myelin glycoprotein (OMgp) antibody, ephrin B3 antibody, semaphorins 4a/4d/6a antibody, netrin 1 antibody, repulsive guidance molecule A (RGMa) antibody, and erythropoietin.

10. A trehalose containing hydrogel comprising:

a first polymer, and

a second polymer; wherein

at least one of the first polymer or the second polymer includes trehalose covalently bound thereto.

11. The trehalose containing hydrogel of embodiment 10, wherein the first polymer includes a trehalose crosslinked thereto.

12. The trehalose containing hydrogel any one or more of embodiments 10-11, wherein the trehalose containing hydrogel is a product of a step growth or chain growth polymerization between: a first polymer component having nucleophilic functional groups, a degree of functionality of greater than or equal to two, and selected from the group consisting of a branched monomer, a multifunctional monomer, a branched polymer, and a multifunctional polymer; and a second polymer component having electrophilic functional groups, a degree of functionality of greater than or equal to two and selected from the group consisting of a branched monomer, a multifunctional monomer, a branched polymer, and a multifunctional polymer.

13. The trehalose containing hydrogel of embodiment 12, wherein the step growth or chain growth polymerization takes place in an aqueous solvent.

14. The trehalose containing hydrogel of any one or more of embodiments 13-14, wherein the first polymer component is a natural or synthetic polymer.

15. The trehalose containing hydrogel of any one or more of embodiments 13-14, wherein the first polymer component includes a multi-arm poly(ethylene glycol) functionalized with terminal thiol groups.

16. The trehalose containing hydrogel of any one or more of embodiments 13-15, wherein first polymer component includes ethoxylated-trimethylolpropan tri(3-mercaptopropionate).

17. The trehalose containing hydrogel of any one or more of embodiments 13-16, wherein the second polymer component includes a trehalose crosslinked thereto.

18. The trehalose containing hydrogel of any one or more of embodiments 13-17, wherein the crosslinked trehalose is derived from one of a methacrylate functionalized trehalose, an ethacrylate functionalized trehalose, a maleimide functionalized trehalose, a vinyl sulfone functionalized trehalose, an acrylate functionalized trehalose, or a thiol functionalized trehalose.

19. The trehalose containing hydrogel of any one or more of embodiments 13-18, wherein the crosslinked trehalose is in the form of a trehalose repeat unit with n repeating units of trehalose.

20. The trehalose containing hydrogel of any one or more of embodiments 13-19, wherein the second polymer component has a terminal functionality selected from the group consisting of methacrylate, ethacrylate, maleimide, and vinyl sulfone or acrylate.

21. The trehalose containing hydrogel of embodiment 19, wherein n is equal to 2 or 3.

22. The trehalose containing hydrogel of any one or more of embodiments 13-21, wherein the second polymer component is selected from the group consisting of trehalose diacrylate and poly(ethylene glycol) diacrylate.

23. The trehalose containing hydrogel of any one or more of embodiments 13-22, wherein a sum of the degree of functionalities of the first polymer component and the second polymer component is greater than or equal to 5.

24. The trehalose containing hydrogel of any one or more of embodiments 13-23, wherein the first polymer component is ethoxylated-trimethylolpropan tri(3-mercaptopropionate), and the second component is trehalose diacrylate.

25. The trehalose containing hydrogel of any one or more of embodiments 10-24 further comprising a third polymer.

26. The trehalose containing hydrogel of embodiment 25, wherein the third component is poly(ethylene glycol diacrylate).

27 The trehalose containing hydrogel of any one or more of embodiments 25-26, wherein the trehalose containing hydrogel is a product of a step growth or chain growth polymerization between: a first polymer component having nucleophilic functional groups, a degree of functionality of greater than or equal to two, and selected from the group consisting of a branched monomer, a multifunctional monomer, a branched polymer, and a multifunctional polymer; a second polymer component having electrophilic functional groups, a degree of functionality of greater than or equal to two and selected from the group consisting of a branched monomer, a multifunctional monomer, a branched polymer, and a multifunctional polymer; and a third polymer component having electrophilic or nucleophilic functional groups, a degree of functionality greater than or equal to one, and selected from the group consisting of a branched monomer, a multifunctional monomer, a branched polymer, and a multifunctional polymer.

28. The trehalose containing hydrogel of embodiment 27, wherein the first component is ethoxylated-trimethylolpropan tri(3-mercaptopropionate), the second component is trehalose diacrylate and the third component is PEG diacrylate.

29. The trehalose containing hydrogel of embodiment 28, wherein, the first component is ethoxylated-trimethylolpropan tri(3-mercaptopropionate), the second component is trehalose diacrylate, and the third component is an 8-arm PEG vinyl sulfone.

30. The trehalose containing hydrogel of any one embodiments 10-29 further comprising a therapeutic.

31. The trehalose containing hydrogel of embodiment 30, wherein the therapeutic is a biological therapeutic.

32. The trehalose containing hydrogel of embodiment 31, wherein the biological therapeutic is selected from the group consisting of a growth factor, a protein, a peptide, an enzyme, an antibody, an RNA, a DNA, a vaccine, and a virus.

33. The trehalose containing hydrogel of any one of embodiments 31-32, wherein the biological therapeutic is selected from the group consisting of chondroitinase ABC (chABC), arylsulfatase B (ARSB), neurotrophin-3 (NT-3), insulin, human growth hormone (HGH), bone morphogenic protein-2 (BMP-2) or related family of BMP's, nerve growth factor (NGF), brain-derived neurotrophic factor (BDNF), glial-cell-line derived neurotrophic factor (GDNF), hepatocyte growth factor (HFG), exozyme C3 transferase (Cethrin) and its derivatives, basic fibroblast growth factor (bFGF), acid fibroblast growth factor (aFGF), transforming growth factor b1 (TGF-β1), epidermal growth factor (EGF), platelet-derived growth factor (PDGF), insulin-like growth factor 1 (IGF-1), vascular endothelial growth factor (VEGF), leukemia inhibitory factor (LIF), and anti-Nogo antibody, myelin associated glycoprotein (MAG) antibody, oligodendrocyte myelin glycoprotein (OMgp) antibody, ephrin B3 antibody, semaphorins 4a/4d/6a antibody, netrin 1 antibody, repulsive guidance molecule A (RGMa) antibody, and erythropoietin.

34. A composition comprising:
a first polymer component having nucleophilic functional groups, a degree of functionality of greater than or equal to two, and is selected from the group consisting of a branched monomer, a multifunctional monomer, a branched polymer, and a multifunctional polymer,
a second polymer component having electrophilic functional groups, a degree of functionality of greater than or equal to two and selected from the group consisting of a branched monomer, a multifunctional monomer, a branched polymer, and a multifunctional polymer,
wherein, at least one of the first polymer component or the second polymer component includes crosslinkable trehalose.

35. The composition of embodiment 34, wherein the first polymer includes a crosslinkable trehalose.

36. The composition of any one of embodiments 34-35, wherein the first polymer component includes a multi-arm poly(ethylene glycol) functionalized with terminal thiol groups.

37. The composition of any one of embodiments 34-35, wherein first polymer component includes ethoxylated-trimethylolpropan tri(3-mercaptopropionate).

38. The composition of any one of embodiments 34-37, wherein the second polymer component includes crosslinkable trehalose.

39. The composition of any one of embodiments 34-38, wherein the crosslinkable trehalose is derived from one of a methacrylate functionalized trehalose, and ethacrylate functionalized trehalose, a maleimide functionalized trehalose, a vinyl sulfone functionalized trehalose, an acrylate functionalized trehalose, or a thiol functionalized trehalose.

40. The composition of embodiment 38, wherein the crosslinkable trehalose is in the form of a trehalose repeat unit with n repeating units of trehalose.

41. The composition of embodiment 40, wherein n is equal to 2 or 3.

42. The composition of any one of embodiments 34-38, wherein the second polymer component is selected from the group consisting of trehalose diacrylate and poly(ethylene glycol) diacrylate.

43. The composition of any one of embodiments 34-42, wherein the second polymer component has a terminal functionality selected from the group consisting of methacrylate, ethacrylate, maleimide, and vinyl sulfone or acrylate.

44. The composition of any one of embodiments 34-43, wherein a sum of the degree of functionalities of the first polymer component and the second polymer component is greater than or equal to 5.

45. The composition of any one of embodiments 34-44, further comprising an aqueous solvent.

46. The composition of any one of embodiments 34-45, wherein the first polymer component is ethoxylated-trimethylolpropan tri(3-mercaptopropionate), and the second component is trehalose diacrylate.

47. The composition of any one of embodiments 34-46 further comprising a third polymer component having electrophilic or nucleophilic functional groups, a degree of functionality greater than or equal to two, and selected from the group consisting of a branched monomer, a multifunctional monomer, a branched polymer, and a multifunctional polymer.

48. The composition of embodiment 47, wherein the third component is poly(ethylene glycol diacrylate).

49. The composition of any one of embodiments 34-47, wherein the first component is ethoxylated-trimethylolpropan tri(3-mercaptopropionate), the second component is trehalose diacrylate and the third component is PEG diacrylate.

50. The composition of any one of embodiments 34-47, wherein, the first component is ethoxylated-trimethylolpropan tri(3-mercaptopropionate), the second component is trehalose diacrylate, and the third component is an 8-arm PEG vinyl sulfone.

51. The composition of any one embodiments 34-50 further comprising a therapeutic.

52. The composition of embodiment 51, wherein the therapeutic is a biological therapeutic.

53. The composition of embodiment 52, wherein the biological therapeutic is selected from the group consisting of a growth factor, a protein, a peptide, an enzyme, an antibody, an RNA, a DNA, a vaccine, and a virus.

54. The composition of any one of embodiments 52-53, wherein the biological therapeutic is selected from the group consisting of chondroitinase ABC (chABC), arylsulfatase B (ARSB), neurotrophin-3 (NT-3), insulin, human growth hormone (HGH), bone morphogenic protein-2 (BMP-2) or related family of BMP's, nerve growth factor (NGF), brain-derived neurotrophic factor (BDNF), glial-cell-line derived neurotrophic factor (GDNF), hepatocyte growth factor (HFG), exozyme C3 transferase (Cethrin) and its derivatives, basic fibroblast growth factor (bFGF), acid fibroblast growth factor (aFGF), transforming growth factor b1 (TGF-β1), epidermal growth factor (EGF), platelet-derived growth factor (PDGF), insulin-like growth factor 1 (IGF-1), vascular endothelial growth factor (VEGF), leukemia inhibitory factor (LIF), and anti-Nogo antibody, myelin associated glycoprotein (MAG) antibody, oligodendrocyte myelin glycoprotein (OMgp) antibody, ephrin B3 antibody, semaphorins 4a/4d/6a antibody, netrin 1 antibody, repulsive guidance molecule A (RGMa) antibody, and erythropoietin.

55. A composition comprising:

a trehalose containing hydrogel, wherein the hydrogel includes a natural or synthetic polymer.

56. The composition of embodiment 55, wherein the trehalose containing hydrogel includes a poly(ethylene glycol) polymer.

57. The composition of any one of embodiments 55-56, wherein the trehalose containing hydrogel includes the product of step growth or chain growth polymerization between ethoxylated-trimethylolpropan tri(3-mercaptopropionate) and trehalose diacrylate.

58. The composition of any one of embodiments 55-56, wherein the trehalose containing hydrogel includes the product of step growth or chain growth polymerization between ethoxylated-trimethylolpropan tri(3-mercaptopropionate), trehalose diacrylate, and PEG diacrylate.

59. The composition of any one of embodiments 55-56, wherein the trehalose containing hydrogel includes the product of step growth or chain growth polymerization between ethoxylated-trimethylolpropan tri(3-mercaptopropionate), trehalose diacrylate, and an 8-arm PEG vinyl sulfone.

60. The composition of any one of embodiments 55-59, further comprising a therapeutic.

61. The composition of embodiment 60, wherein the therapeutic is a biological therapeutic.

62. The composition of any one of embodiments 60-61, wherein the biological therapeutic is selected from the group consisting of a growth factor, a protein, peptide, an enzyme, an antibody, an RNA, a DNA, a vaccine, and a virus.

63. The composition of any one of embodiments 60-62, wherein the biological therapeutic is selected from the group consisting of chondroitinase ABC (chABC), arylsulfatase B (ARSB), neurotrophin-3 (NT-3), insulin, human growth hormone (HGH), bone morphogenic protein-2 (BMP-2) or related family of BMP's, nerve growth factor (NGF), brain-derived neurotrophic factor (BDNF), glial-cell-line derived neurotrophic factor (GDNF), hepatocyte growth factor (HFG), exozyme C3 transferase (Cethrin) and its derivatives, basic fibroblast growth factor (bFGF), acid fibroblast growth factor (aFGF), transforming growth factor b1 (TGF-β1), epidermal growth factor (EGF), platelet-derived growth factor (PDGF), insulin-like growth factor 1 (IGF-1), vascular endothelial growth factor (VEGF), leukemia inhibitory factor (LIF), and anti-Nogo antibody, myelin associated glycoprotein (MAG) antibody, oligodendrocyte myelin glycoprotein (OMgp) antibody, ephrin B3 antibody, semaphorins 4a/4d/6a antibody, netrin 1 antibody, repulsive guidance molecule A (RGMa) antibody, and erythropoietin.

64. A kit comprising
a first vessel containing:
a first polymer component having nucleophilic functional groups, a degree of functionality of greater than or equal to two, and is selected from the group consisting of a branched monomer, a multifunctional monomer, a branched polymer, and a multifunctional polymer, and
a second vessel containing:
a second polymer component having electrophilic functional groups, a degree of functionality of greater than or equal to two and selected from the group consisting of a branched monomer, a multifunctional monomer, a branched polymer, and a multifunctional polymer;
wherein, at least one of the first polymer component or the second polymer component includes crosslinkable trehalose.

65. The kit of embodiment 61 further comprising a therapeutic in at least one of the first container, the second container, or a third container.

66. The kit of any one of embodiments 61-65 further comprising a third vessel containing a third polymer component having electrophilic or nucleophilic functional groups, a degree of functionality greater than or equal to one, and selected from the group consisting of a branched monomer, a multifunctional monomer, a branched polymer, and a multifunctional polymer.

67. The kit of one of embodiments 61-65 further comprising directions on the making of a trehalose containing hydrogel utilizing the contents of the kit.

68. A method of controlled therapeutic delivery comprising:

implanting the trehalose containing hydrogel or composition of any one of embodiments 1-63 at a treatment site in a patient in need thereof.

69. The method of controlled therapeutic delivery embodiment 68, wherein implanting includes placing the trehalose containing hydrogel at the treatment site.

70. The method of controlled therapeutic delivery embodiment 68, wherein implanting includes injecting precursors of the trehalose containing hydrogel at the treatment site.

71. The method of controlled therapeutic delivery embodiment 68, wherein implanting includes injecting the composition of any one of embodiments 34-62 at the treatment site.

72. A method of controlled therapeutic delivery comprising: injecting the contents of one or more of the vessels of the kit of any one of embodiments 64-67 to a treatment site in a patient in need thereof.

73. A method of controlled therapeutic delivery comprising: forming a trehalose containing hydrogel from the contents of one or more of the vessels of the kit of any one of embodiments 64-67, and implanting the trehalose containing hydrogel at a treatment site in a patient in need thereof.

74. A method of making a therapeutic implant comprising: inserting a crosslinkable trehalose within a synthetic polymeric hydrogel to form a crosslinked hydrogel; and incorporating a therapeutic in the crosslinked hydrogel to form the therapeutic implant.

75. A method of making a trehalose containing hydrogel comprising forming the composition of any one of embodiments 34-62, or combining the contents of one or more of the vessels of the kit of any one or more of embodiments 64-74.

76. Any of the preceding embodiments where “trehalose” is replaced by “sugar.”

77. Embodiment 76, wherein the sugar is selected from monosaccharides, disaccharides, oligosaccharides, polysaccharides, sucrose, glucose, mannose, galactose, fructose, oligosaccharides, oligosaccarides containing a mixture of sugars listed herein, chitosan, dextran, alginate, hyaluronic acid, cellulose (non-modified or modified), amylose, or chondroitin sulfate.

Further embodiments herein may be formed by supplementing an embodiment with one or more element from any one or more other embodiment herein, and/or substituting one or more element from one embodiment with one or more element from one or more other embodiment herein.

### EXAMPLES

The following non-limiting examples are provided to illustrate particular embodiments. The embodiments throughout may be supplemented with one or more detail from one or more example below, and/or one or more element from an embodiment may be substituted with one or more detail from one or more example below.

### Example 1

### Synthesis of Trehalose Diacrylate

Enzymatic, chemoselective modification of trehalose to form trehalose diacrylate has been reported. See, John et al., 2006 and Zhu and Dordick, 2006, cited below, which are incorporated herein by reference as if fully set forth. An exemplary functional monomer was synthesized by reacting a four-mole excess of vinyl acrylate (CAS: 2177-18-6) with trehalose dihydrate in the presence of Novozyme 435 (Lipase B from Candida antarctica that is attached to acrylic resin) in dry acetone at 50° C. and agitated conditions (either by orbital shaker or magnetic stir plate) for 48 hours. Hydroquinone monomethyl ether (MEHQ) and butylated hydroxytoluene (BHT) were used to prevent excessive radical homopolymerizaiton of the product. The crude product was purified via silica flash chromatography on an ISCO Combiflash system using ethyl acetate and 80% methanol as the binary solvent system. Product was characterized using LC-MS (FIG. 2) and ¹H-NMR (FIG. 3). See Reaction Scheme 3, below.

[chemical structure]

### Example 2

### Synthesis of Multifunctional Thiol Trehalose

To synthesize a multifunctional thiol trehalose a two step reaction scheme involving a divinyl ester intermediate was applied. The first step of the synthesis involved reacting trehalose dihydrate with a twelve mole excess of a divinyl ester crosslinker such as divinyl adipate or divinyl sebacate in the presence of Novozyme 435 (Lipase B from Candida antarctica that is attached to acrylic resin) in acetone at 50° C. under agitated conditions for 48 hours. The reaction mixture was filtered to remove the lipase resin and then purified by precipitating three times in cold hexane. The purified trehalose divinyl ester was collected by filtration. The synthesis of this divinyl ester trehalose has been reported previously as a monomer for making linear ployesters. See Park, O.-J.; Kim, D.-Y.; Dordick, J. S. Enzyme-catalyzed synthesis of sugar-containing monomers and linear polymers. Biotechnol. Bioeng. 2000, 70, 208-216, which is incorporated herein by reference as if fully set forth. To synthesize the multifunctionalized thiol trehalose, the purified trehalose divinyl ester was reacted with a thiol containing primary alcohol. The thiol containing primary alcohol could include one or more of 2,3-dimercapto-1-propanol, 2-mercaptoethanol, or 1-thioglycerol. As an example, a 4 mole excess of 2,3-dimercapto-1-propanol was allowed to react with trehalose divinyl ester in the presence of Novozyme 435 (Lipase B from Candida antarctica that is attached to acrylic resin) in acetone at 50° C. for 24 hours under inert conditions. After 24 hours the reaction was stopped by filtering off the Lipase B resin and the tetra functionalized thiol containing trehalose is purified by precipitating three times in ethyl ether. Purified tetra functionalized thiol trehalose was collected by filtering off the ethyl ether after the final precipitation step. Product was characterized using LC-MS (FIG. 5) and 1H-NMR (FIG. 6). See Reaction Scheme 4, below.

[chemical structure]

### Example 3

### NT-3 Stabilization Using Crosslinkable Trehalose

To determine whether the stabilizing capacity of trehalose was altered following diacrylate or dimethacrylate modification of the sugar via the enzymatic catalyzed assay, a stabilization assay was performed. Using the neurotrophin NT-3 as a model protein, solutions of the protein were prepared that compared the stabilizing capacity of trehalose alone with that of the two modified sugars. A solution containing no trehalose was used as a control. The NT-3 solutions were incubated at 37° C. for defined periods of time, namely 2 hours, 3, 7 14 and 21 days. At each time point triplicate samples of each group were assayed for activity using an NT-3 ELISA assay (R&D Systems). Percentage activity was computed by comparing the ELISA concentration observed with the known original starting concentration.

### Example 4

### Preparation of Trehalose Hydrogels Crosslinked by Trehalose Diacrylate and Thiol-Containing 4-Arm PEG Polymer

Hydrogels formed by mixing trehalose diacrylate and thiol-containing 4-arm PEG of molecular weight 5000 (4arm-PEG5k-SH). Briefly, trehalose diacrylate was dissolved in PBS (pH7.4, 1.06 mM potassium phosphate monobasic, 150 mM sodium chloride, 2.97 mM sodium phosphate dibasic) to prepare a trehalose-containing precursor solution at a concentration of 50 mg/ml. 9.42 mg 4arm-PEG5k-SH (0.0075 mmol thiols) was dissolved in 66.1 μl PBS and the solution was mixed with 33.9 μl trehalose diacrylate solution (1.7 mg, 1 molar equivalents based on thiols) and by vortexing. Trehalose diacrylate and 4arm-PEG5k-SH are soluble in aqueous buffer and the mixture solution formed clear solid gel with polymer content of 10 wt % within several minutes at room temperature. Mechanical property and gelling time were characterized by rheometer as shown in FIG. 8.

### Example 5

### Covalent Incorporation of Trehalose into Hydrogels Crosslinked by PEG diacrylate and 4arm-PEG5k-SH

Hydrogels formed by mixing 4arm-PEG5k-SH, PEG diacrylate, and varying amounts of trehalose diacrylate. Briefly, as an example, trehalose diacrylate was dissolved in PBS to prepare a trehalose-containing precursor solution at a concentration of 50 mg/ml. 1.05 mg PEG diacrylate of molecular weight 575 (PEGDA575, 0.5 molar equivalents based on thiols) was dissolved in 40.8 μl PBS and the solution was mixed with 16.4 μl Trehalose diacrylate (0.82 mg, 0.5 molar equivalents based on thiols) solution. The acrylate-containing mixture solution was then mixed with 41.8 μl PBS solution containing 9.2 mg 4arm-PEG5k-SH (0.0073 mmol thiol). A clear solid gel with polymer content of 10 wt % was formed within several minutes at room temperature. Mechanical property and gelling time were characterized by rheometer as shown in FIG. 8.

### Example 6

### Covalent Incorporation of Trehalose into Hydrogels Crosslinked by PEG Diacrylate and Thiol-Containing 3-Arm PEG Polymer

Hydrogels formed by mixing ethoxylated-trimethylolpropan tri(thioglycolate) (TMPE-TGA), PEG diacrylate, and varying amounts of trehalose diacrylate. The following protocol has been used for gel formulation at the 150 μl scale. Briefly, TMPE-TGA was dissolved in PBS to prepare a 40 wt % thiol-containing precursor solution. Trehalose diacrylate was dissolved in PBS to prepare a trehalose-containing precursor solution at a concentration of 40 mg/ml. 10.875 mg PEG diacrylate of molecular weight 700 (PEGDA700, 0.75 molar equivalents based on thiols) was dissolved in 49.94 μl PBS and the solution was mixed with 58.3 μl trehalose diacrylate solution (2.33 mg, 0.25 molar equivalents based on thiols) and 41.8 μl TMPE-TGA solution (0.041 mmol thiols) by vortexing. TMPE-TGA, trehalose diacrylate and PEG diacrylate are all water soluble and the mixture solution formed clear solid gel with polymer content of 20 wt % within several minutes at room temperature.

Likewise, a gel of higher amount of trehalose was prepared by first dissolving 8.91 mg PEGDA700 (0.6 molar equivalents based on thiols) in 4.4 μl PBS followed by mixing this solution with 95.5 μl trehalose diacrylate solution (3.82 mg, 0.4 molar equivalents based on thiols) and 42.8 μl TMPE-TGA solution (0.042 mmol thiols). A clear solid gel with polymer content of 20 wt % formed within several minutes at room temperature.

The above 2 gels were swollen in PBS at 37° C. and the wet gel weight measurements were made post 24 hours incubation. Gels were also weighted wet after 48 hours incubation with no significant increase in wet masses observed. The swelling ratios were calculated based on the equation (M_{swell}/M_{cure})×100% and determined as 142.8% for gels with trehalose diacrylate of 0.25 molar equivalents based on thiols, and 170.6% for gels with 0.4 molar equivalents trehalose diacrylate.

The above protocol has also been used to form hydrogels with ETTMP, PEG diacrylate, and varying amounts of trehalose diacrylate. Trehalose containing hydrogels formed with 3-arm PEG thiols were characterized using FTIR (FIG. 7), rheology (FIG. 9), and swelling (FIG. 10).

### Example 7

### Covalent Incorporation of Trehalose into Non-Swelling Hydrogels Crosslinked by TMPE-TGA and Vinyl Sulfone-Containing Multi-Arm PEG Polymer

Hydrogels formed by mixing TMPE-TGA, vinyl sulfone-containing 8-arm PEG of molecular weight 10000 (8arm-PEG10k-VS), and varying amounts of trehalose diacrylate. Briefly, TMPE-TGA was dissolved in PBS to prepare a 40 wt % thiol-containing precursor solution. Trehalose diacrylate was dissolved in PBS to prepare a trehalose-containing precursor solution at a concentration of 40 mg/ml. 5.2 mg 8arm-PEG10k-VS (0.75 molar equivalents based on thiols) was dissolved in 136.8 μl PBS and the solution was added to 7.7 μl trehalose diacrylate solution (0.31 mg, 0.25 molar equivalents based on thiols) followed by mixing with 5.5 μl 40 wt % TMPE-TGA solution (0.0054 mmol thiols). TMPE-TGA, trehalose diacrylate and PEG diacrylate are all water soluble and the mixture solution formed clear solid gel with polymer content of 5 wt % within several minutes at room temperature.

The yielded gels were swollen in PBS at 37° C. and the wet gel weight measurements were made post 24 hours incubation. Gels were also weighted wet after 48 hours incubation with no significant increase in wet masses observed. The swelling ratios were determined as 109.8% by calculating based on the equation [(M_{swell}/M_{cure})−1]×100%.

The above protocol has also been used to form hydrogels with TMPE-TGA, vinyl sulfone-containing 4-arm PEG of molecular weight 10,000 Da (4arm-PEG10k-VS), and varying amounts of trehalose diacrylate.

### Example 8

### Preparation of Protein-Loaded Trehalose Hydrogel

Protein was loaded within hydrogels that formed by mixing TMPE-TGA, PEG diacrylate, and varying amounts of trehalose diacrylate. For example, TMPE-TGA was dissolved in PBS to prepare a 40 wt % thiol-containing precursor solution. 43.9 μl TMPE-TGA(0.044 mmol thiols) solution was gently mixed with 20 μl chondroitinase ABC (cABC, 10 unit/μl) by pipetting up and down to yield solution A. Trehalose diacrylate was dissolved in PBS to prepare a trehalose-containing precursor solution at a concentration of 100 mg/ml. 9.2 mg PEGDA700 (0.6 molar equivalents based on thiols) was dissolved in 39.4 μl PBS and the solution was added to 39.2 μl trehalose diacrylate solution (3.9 mg, 0.4 molar equivalents based on thiols) to yield acrylate-containing solution B. cABC-loaded trehalose hydrogel with polymer content of 20 wt % formed within several minutes by mixing solution A and B at room temperature.

For another example, protein-loaded hydrogels were prepared by mixing TMPE-TGA, 8arm-PEG10k-VS, and varying amounts of trehalose diacrylate. Briefly, TMPE-TGA was dissolved in PBS to prepare a 40 wt % thiol-containing precursor solution. 5.5 μl TMPE-TGA (0.0055 mmol thiols) solution was gently mixed with 20 μl chondroitinase ABC (cABC, 10 unit/μl) by pipetting up and down to yield solution A. Trehalose diacrylate was dissolved in PBS to prepare a trehalose-containing precursor solution at a concentration of 40 mg/ml. 5.2 mg 8arm-PEG10k-VS (0.75 molar equivalents based on thiols) was dissolved in 116.8 μl PBS and the solution was added to 7.7 μl trehalose diacrylate solution (0.31 mg, 0.25 molar equivalents based on thiols) to yield solution B. cABC-loaded trehalose hydrogel with polymer content of 5 wt % formed within several minutes by mixing solution A and B at room temperature.

Likewise, a protein-loaded gel with higher polymer content was prepared by mixing TMPE-TGA, 8arm-PEG10k-VS, and varying amounts of trehalose diacrylate. Briefly, TMPE-TGA was dissolved in PBS to prepare a 40 wt % thiol-containing precursor solution. 11. 5 μl TMPE-TGA (0.011 mmol thiols) solution was gently mixed with 20 μl chondroitinase ABC (cABC, 10 unit/μl) by pipetting up and down to yield solution A. Trehalose diacrylate was dissolved in PBS to prepare a trehalose-containing precursor solution at a concentration of 100 mg/ml. 10.7 mg 8arm-PEG10k-VS (0.75 molar equivalents based on thiols) was dissolved in 102.1 μl PBS and the solution was added to 6.4 μl trehalose diacrylate solution (0.64 mg, 0.25 molar equivalents based on thiols) to yield solution B. cABC-loaded trehalose hydrogel with polymer content of 10 wt % formed within several minutes by mixing solution A and B at room temperature.

### Example 9

### Enzyme Thermostabilization in Trehalose Containing Hydrogels

Horseradish peroxidase (HRP) is an important enzyme, which has been widely used in biotechnology and bioremediation. However, the thermostability of HRP limits its industrial applications. Herein, HRP was used as a model protein to evaluate the effects of hydrogels containing varying amount of trehalose on protein stabilization. The activity of HRP after exposure to 70° C. for 2 hours within hydrogels or in solution was analyzed. HRP-Loaded hydrogels containing varying amount of trehalose were prepared using 4arm-PEG5k-SH, PEGDA575 and trehalose diacrylate as described above.

Typically, HRP (2 mg/ml) solution was prepared by adding HRP into pH7.4 PBS. 37.7 mg 4arm-PEG5k-SH (0.0301 mmol thiols) was dissolved in 224.4 μl PBS and the solution was gently mixed with 40 μl HRP solution (2 mg/ml) by pipetting up and down to yield solution A. Trehalose diacrylate was dissolved in PBS to prepare a trehalose-containing precursor solution at a concentration of 50 mg/ml. 135.6 μl trehalose diacrylate solution (6.8 mg, 1 molar equivalents based on thiols) was added to solution A by pipetting up and down. This protocol has been used for the formation of gels with polymer content of 10 wt % at the 400 μl scale. The resulting mixture solution was aliquoted to PTFE mold (0.8 cm in diameter) at 100 μl per well. Clear solid gel containing HRP (20 μg for each gel slab) formed within several minutes at room temperature and was designated as PEG-Trehalose100.

22.9 mg 4arm-PEG5k-SH (0.0183 mmol thiols) was dissolved in 79.5 μl PBS and the solution was gently mixed with 25 μl HRP solution (2 mg/ml) by pipetting up and down to yield solution A. Trehalose diacrylate was dissolved in PBS to prepare a trehalose-containing precursor solution at a concentration of 50 mg/ml. 2.6 mg PEGDA575 (0.5 molar equivalents based on thiols) was dissolved in 102.1 μl PBS and the solution was added to 41.1 μl trehalose diacrylate solution (2.1 mg, 0.5 molar equivalents based on thiols) to yield acrylate-containing solution B. Solution B was added to solution A by pipetting up and down. This protocol has been used for the formation of gels with polymer content of 10 wt % at the 250 μl scale. The resulting mixture solution was aliquoted to PTFE mold (0.8 cm in diameter) at 100 μl per well. Clear solid gel containing HRP (20 μg for each gel slab) formed within several minutes at room temperature and was designated as PEG-Trehalose50.

22.2 mg 4arm-PEG5k-SH (0.0177 mmol thiols) was dissolved in 100 μl PBS and the solution was gently mixed with 25 μl HRP solution (2 mg/ml) by pipetting up and down to yield solution A. 5.1 mg PEGDA575 (1 molar equivalents based on thiols) was dissolved in 120.4 μl PBS and this solution was added to solution A by pipetting up and down. This protocol has been used for the formation of gels with polymer content of 10 wt % at the 250 μl scale. The resulting mixture solution was aliquoted to PTFE mold (0.8 cm in diameter) at 100 μl per well. Clear solid gel containing HRP (20 μg for each gel slab) formed within several minutes at room temperature and was designated as PEG-Trehalose0.

Non-modified trehalose dehydrate was mixed with HRP solution to give a final concentration of 200 μg/ml of HRP. Trehalose dihydrate was added to be equivalent to the trehalose molar amount in the above hydrogel formulations, respectively. Solution and hydrogel samples were incubated at 70° C. for 2 hours, and a nonheated HRP solution (control) was stored at 4° C. until the activity assay was performed. After exposure to heating, hydrogel samples were equilibrated to room temperature and manually smashed using a spatulas followed by incubation at 37° C. for 20 hours to accelerate HRP release. The heated HRP solutions were also incubated at 37° C. for 20 hours. Bradford assay was performed to determine HRP concentration in collected samples. 3,3′,5,5′-tetramethylbenzidine (TMB) was used as a substrate and 2 M sulfuric acid as the stop solution to determine HRP activity by absorption at 450 nm. The activity of per mg protein in heated samples was normalized against nonheated HRP solution control.

Activity of HRP (200 μg/ml) after 2 hours incubation at 70° C., within solutions or hydrogels containing varying amount of trehalose. For solution samples, non-modified trehalose dehydrate was added to be equivalent to the molar amounts of trehalose diacrylate in their corresponding hydrogels. Data shown as the average of n=2 with standard deviation.

The trehalose containing hydrogel demonstrated superior preservation of HRP activity upon exposure to extreme temperature environments (FIG. 12).

### REFERENCES

[1] Stanwick, J. C., Baumann, M. D., Shoichet, M. S. Enhanced neurotrophin-3 bioactivity and release from a nanoparticle-loaded composite hydrogel. J. Control Release 160 (2012) 666-675.
[2] Lee, H., McKeon, R. J., Bellamkonda, R. V. Sustained delivery of thermostabilized chABC enhances axonal sprouting and functional recovery after spinal cord injury. Proc. Natl. Acad. Sci. USA 107 (2010) 3340-3345.
[3] John, G., Zhu, G., Li, J., Dordick, J. S. Enzymatically derived sugar-containing self-assembled organogels with nanostructured morphologies. Angew. Chem. Int. Ed. 45 (2006) 4772-4775.
[4] Zhu, G., and Dordick, J. S. Solvent Effect on Organogel Formation by Low Molecular Weight Molecules. Chem. Mater. 18 (2006) 5988-5995.
[5] Dordick, J. S., Rethwisch, D. G., Patil, D. R., Martin, B. D., Linhardt, R. J. Sugar-based polymers. U.S. Pat. No. 5,854,030 Dec. 29, 1998.
[6] Park, O.-J.; Kim, D.-Y.; Dordick, J. S. Enzyme-catalyzed synthesis of sugar-containing monomers and linear polymers. Biotechnol. Bioeng. 2000, 70, 208-216.

The references cited throughout this application are incorporated for all purposes apparent herein and in the references themselves as if each reference was fully set forth. For the sake of presentation, specific ones of these references are cited at particular locations herein. A citation of a reference at a particular location indicates a manner(s) in which the teachings of the reference are incorporated. However, a citation of a reference at a particular location does not limit the manner in which all of the teachings of the cited reference are incorporated for all purposes.

It is understood, therefore, that this invention is not limited to the particular embodiments disclosed, but is intended to cover all modifications which are within the spirit and scope of the invention as defined by the appended claims; the above description; and/or shown in the attached drawings.

What is claimed is:

1. A composition comprising:
a trehalose containing hydrogel; and
a therapeutic.

2. The composition of claim 1, wherein the trehalose containing hydrogel includes a natural or synthetic polymer.

3. The composition of claim 1, wherein the trehalose is in the form of a trehalose repeat unit with n repeating units of trehalose and n is greater than or equal to two.

4. The composition of claim 1, wherein the therapeutic is a biological therapeutic.

5. The composition of claim 4, wherein the biological therapeutic is selected from the group consisting of a growth factor, a protein, a peptide, an enzyme, an antibody, an RNA, a DNA, a vaccine, and a virus.

6. The composition of claim 5, wherein the biological therapeutic is selected from the group consisting of chondroitinase ABC (chABC), arylsulfatase B (ARSB), neurotrophin-3 (NT-3), insulin, human growth hormone (HGH), bone morphogenic protein-2 (BMP-2) or related family of BMP's, nerve growth factor (NGF), brain-derived neurotrophic factor (BDNF), glial-cell-line derived neurotrophic factor (GDNF), hepatocyte growth factor (HFG), exozyme C3 transferase (Cethrin) and its derivatives, basic fibroblast growth factor (bFGF), acid fibroblast growth factor (aFGF), transforming growth factor b1 (TGF-β1), epidermal growth factor (EGF), platelet-derived growth factor (PDGF), insulin-like growth factor 1 (IGF-1), vascular endothelial growth factor (VEGF), leukemia inhibitory factor (LIF), and anti-Nogo antibody, myelin associated glycoprotein (MAG) antibody, oligodendrocyte myelin glycoprotein (OMgp) antibody, ephrin B3 antibody, semaphorins 4a/4d/6a antibody, netrin 1 antibody, repulsive guidance molecule A (RGMa) antibody, and erythropoietin.

7. A trehalose containing hydrogel comprising:
a first polymer, and
a second polymer; wherein
at least one of the first polymer or the second polymer includes trehalose covalently bound thereto.

8. The trehalose containing hydrogel of claim 7, wherein the first polymer includes a trehalose crosslinked thereto.

9. The trehalose containing hydrogel of claim 7, wherein
the trehalose containing hydrogel is a product of a step growth or chain growth polymerization between: a first polymer component having nucleophilic functional groups, a degree of functionality of greater than or equal to two, and selected from the group consisting of a branched monomer, a multifunctional monomer, a branched polymer, and a multifunctional polymer; and a second polymer component having electrophilic functional groups, a degree of functionality of greater than or equal to two and selected from the group consisting of a branched monomer, a multifunctional monomer, a branched polymer, and a multifunctional polymer.

10. The trehalose containing hydrogel of claim 9, wherein the step growth or chain growth polymerization takes place in an aqueous solvent.

11. The trehalose containing hydrogel of claim 9, wherein the first polymer component is a natural or synthetic polymer.

12. The trehalose containing hydrogel of claim 9, wherein the second polymer component includes a trehalose crosslinked thereto.

13. The trehalose containing hydrogel of claim 12, wherein the crosslinked trehalose is derived from one of a methacrylate functionalized trehalose, and ethacrylate functionalized trehalose, a maleimide functionalized trehalose, or a vinyl sulfone functionalized trehalose or an acrylate functionalized trehalose.

14. The trehalose containing hydrogel of claim 12, wherein the crosslinked trehalose is in the form of a trehalose repeat unit with n repeating units of trehalose.

15. The trehalose containing hydrogel of claim 14, wherein the second polymer component has a terminal functionality selected from the group consisting of methacrylate, ethacrylate, maleimide, and vinyl sulfone or acrylate.

16. The trehalose containing hydrogel of claim 14, wherein n is equal to 2 or 3.

17. The trehalose containing hydrogel of claim 12, wherein the second polymer component is selected from the group consisting of trehalose diacrylate and poly(ethylene glycol) diacrylate.

18. The trehalose containing hydrogel of claim 12, wherein a sum of the degree of functionalities of the first polymer component and the second polymer component is greater than or equal to 5.

19. The trehalose containing hydrogel of claim 7 further comprising a third polymer.

20. The trehalose containing hydrogel of claim 19, the trehalose containing hydrogel is a product of a step growth or chain growth polymerization between: a first polymer component having nucleophilic functional groups, a degree of functionality of greater than or equal to two, and selected from the group consisting of a branched monomer, a multifunctional monomer, a branched polymer, and a multifunctional polymer; a second polymer component having electrophilic functional groups, a degree of functionality of greater than or equal to two and selected from the group consisting of a branched monomer, a multifunctional monomer, a branched polymer, and a multifunctional polymer; and a third polymer component having electrophilic or nucleophilic functional groups, a degree of functionality greater than or equal to one, and selected from the group consisting of a branched monomer, a multifunctional monomer, a branched polymer, and a multifunctional polymer.

21. The trehalose containing hydrogel of claim 7 further comprising a therapeutic.

22. The trehalose containing hydrogel of claim 21, wherein the therapeutic is a biological therapeutic.

23. The trehalose containing hydrogel of claim 22, wherein the biological therapeutic is selected from the group consisting of a growth factor, a protein, an enzyme, a peptide, an antibody, an RNA, a DNA, a vaccine, and a virus.

24. The trehalose containing hydrogel of claim 23, wherein the biological therapeutic is selected from the group consisting of chondroitinase ABC (chABC), arylsulfatase B (ARSB), neurotrophin-3 (NT-3), insulin, human growth hormone (HGH), bone morphogenic protein-2 (BMP-2) or related family of BMP's, nerve growth factor (NGF), brain-derived neurotrophic factor (BDNF), glial-cell-line derived neurotrophic factor (GDNF), hepatocyte growth factor (HFG), exozyme C3 transferase (Cethrin) and its derivatives, basic fibroblast growth factor (bFGF), acid fibroblast growth factor (aFGF), transforming growth factor b1 (TGF-β1), epidermal growth factor (EGF), platelet-derived growth factor (PDGF), insulin-like growth factor 1 (IGF-1), vascular endothelial growth factor (VEGF), leukemia inhibitory factor (LIF), and anti-Nogo antibody, myelin associated glycoprotein (MAG) antibody, oligodendrocyte myelin glycoprotein (OMgp) antibody, ephrin B3 antibody, semaphorins 4a/4d/6a antibody, netrin 1 antibody, repulsive guidance molecule A (RGMa) antibody, and erythropoietin.

25. A composition comprising:
a first polymer component having nucleophilic functional groups, a degree of functionality of greater than or equal to two, and is selected from the group consisting of a branched monomer, a multifunctional monomer, a branched polymer, and a multifunctional polymer,
a second polymer component having electrophilic functional groups, a degree of functionality of greater than or equal to two and selected from the group consisting of a branched monomer, a multifunctional monomer, a branched polymer, and a multifunctional polymer,
wherein, at least one of the first polymer component or the second polymer component includes crosslinkable trehalose.

26. The composition of claim 25, wherein the first polymer incudes a crosslinkable trehalose.

27. The composition of claim 25, wherein the first polymer component includes a multi-arm poly(ethylene glycol) functionalized with terminal thiol groups.

28. The composition of claim 25, wherein first polymer component includes ethoxylated-trimethylolpropan tri(3-mercaptopropionate).

29. The composition of claim 25, wherein the second polymer component includes a crosslinked trehalose.

30. The composition of claim 29, wherein the crosslinkable trehalose is derived from one of a methacrylate functionalized trehalose, and ethacrylate functionalized trehalose, a maleimide functionalized trehalose, or a vinyl sulfone functionalized trehalose or an acrylate functionalized trehalose.

31. The composition of claim 29, wherein the crosslinkable trehalose is in the form of a trehalose repeat unit with n repeating units of trehalose.

32. The composition of claim 40, wherein n is equal to 2 or 3.

33. The composition of claim 29, wherein the second polymer component is selected from the group consisting of trehalose diacrylate and poly(ethylene glycol) diacrylate.

34. The composition of claim 25, wherein the first polymer component has a terminal functionality selected from the group consisting of methacrylate, ethacrylate, maleimide, and vinyl sulfone or acrylate.

35. The composition of claim 25, wherein a sum of the degree of functionalities of the first polymer component and the second polymer component is greater than or equal to 5.

36. The composition of claim 25, further comprising an aqueous solvent.

37. The composition of claim 25, wherein the first polymer component is ethoxylated-trimethylolpropan tri(3-mercaptopropionate), and the second component is trehalose diacrylate.

38. The composition of claim 25 further comprising a third polymer component having electrophilic or nucleophilic functional groups, a degree of functionality greater than or equal to two, and selected from the group consisting of a branched monomer, a multifunctional monomer, a branched polymer, and a multifunctional polymer.

39. The composition of claim 25 further comprising a therapeutic.

40. The composition of claim 39, wherein the therapeutic is a biological therapeutic.

41. The composition of claim 40, wherein the biological therapeutic is selected from the group consisting of a growth factor, a protein, a peptide, an enzyme, an antibody, an RNA, a DNA, a vaccine, and a virus.

42. The composition of claim 41, wherein the biological therapeutic is selected from the group consisting of chondroitinase ABC (chABC), arylsulfatase B (ARSB), neurotrophin-3 (NT-3), insulin, human growth hormone (HGH), bone morphogenic protein-2 (BMP-2) or related family of BMP's, nerve growth factor (NGF), brain-derived neurotrophic factor (BDNF), glial-cell-line derived neurotrophic factor (GDNF), hepatocyte growth factor (HFG), exozyme C3 transferase (Cethrin) and its derivatives, basic fibroblast growth factor (bFGF), acid fibroblast growth factor (aFGF), transforming growth factor b1 (TGF-β1), epidermal growth factor (EGF), platelet-derived growth factor (PDGF), insulin-like growth factor 1 (IGF-1), vascular endothelial growth factor (VEGF), leukemia inhibitory factor (LIF), and anti-Nogo antibody, myelin associated glycoprotein (MAG) antibody, oligodendrocyte myelin glycoprotein (OMgp) antibody, ephrin B3 antibody, semaphorins 4a/4d/6a antibody, netrin 1 antibody, repulsive guidance molecule A (RGMa) antibody, and erythropoietin.

43. A composition comprising:
a trehalose containing hydrogel, wherein the hydrogel includes a natural or synthetic polymer.

44. The composition of claim 43, wherein the trehalose containing hydrogel includes a poly(ethylene glycol) polymer.

45. The composition of claim 43 further comprising a therapeutic.

46. The composition of claim 45, wherein the therapeutic is a biological therapeutic.

47. The composition of claim 46, wherein the biological therapeutic is selected from the group consisting of a growth factor, a protein, peptide, an enzyme, an antibody, an RNA, a DNA, a vaccine, and a virus.

48. The composition of claim 47, wherein the biological therapeutic is selected from the group consisting of chondroitinase ABC (chABC), arylsulfatase B (ARSB), neurotrophin-3 (NT-3), insulin, human growth hormone (HGH), bone morphogenic protein-2 (BMP-2) or related family of BMP's, nerve growth factor (NGF), brain-derived neurotrophic factor (BDNF), glial-cell-line derived neurotrophic factor (GDNF), hepatocyte growth factor (HFG), exozyme C3 transferase (Cethrin) and its derivatives, basic fibroblast growth factor (bFGF), acid fibroblast growth factor (aFGF), transforming growth factor b1 (TGF-β1), epidermal growth factor (EGF), platelet-derived growth factor (PDGF), insulin-like growth factor 1 (IGF-1), vascular endothelial growth factor (VEGF), leukemia inhibitory factor (LIF), and anti-Nogo antibody, myelin associated glycoprotein (MAG) antibody, oligodendrocyte myelin glycoprotein (OMgp) antibody, ephrin B3 antibody, semaphorins 4a/4d/6a antibody, netrin 1 antibody, repulsive guidance molecule A (RGMa) antibody, and erythropoietin.

49. A kit comprising
a first vessel containing:
a first polymer component having nucleophilic functional groups, a degree of functionality of greater than or equal to two, and is selected from the group consisting of a branched monomer, a multifunctional monomer, a branched polymer, and a multifunctional polymer, and
a second vessel containing:
a second polymer component having electrophilic functional groups, a degree of functionality of greater than or equal to two and selected from the group consisting of a branched monomer, a multifunctional monomer, a branched polymer, and a multifunctional polymer;
wherein, at least one of the first polymer component or the second polymer component includes crosslinkable trehalose.

50. The kit of claim 49 further comprising a therapeutic in at least one of the first container, the second container, or a third container.

51. The kit of claim 46 further comprising a third vessel containing a third polymer component having electrophilic or nucleophilic functional groups, a degree of functionality greater than or equal to two, and selected from the group consisting of a branched monomer, a multifunctional monomer, a branched polymer, and a multifunctional polymer.

52. The kit of claim 49 further comprising directions on the making of a trehalose containing hydrogel utilizing the contents of the kit.

53. A method of controlled therapeutic delivery comprising:
implanting the trehalose containing hydrogel claim 1 or precursors thereof at a treatment site in a patient in need thereof.

54. The method of controlled therapeutic delivery claim 53, wherein implanting includes injecting the precursors of the trehalose containing hydrogel at the treatment site.

55. The method of controlled therapeutic delivery claim 53, wherein the at least one treatment site of administration is selected from the group consisting of spinal cord pia mater of the patient, arachnoid mater of the patient, intrathecal portions of spinal nerves of the patient, epidural, and directly to spinal cord parenchyma of the patient.

56. The method of controlled therapeutic delivery of claim 55, wherein the therapeutic is chondroitinase ABC, neurotrophin-3, or exozyme C3 transferase.

57. A method of making a therapeutic implant comprising: inserting a crosslinkable trehalose polymer within a synthetic polymeric hydrogel to form a crosslinked hydrogel; and incorporating a therapeutic in the crosslinked hydrogel to form the therapeutic implant.
